(19) United States
(12) Patent Application Publication
(10) Pub. No.: US 2012/0157878 A1
(43) Pub. Date: Jun. 21, 2012

(54) ANATOMICALLY DESIGNED AND COLLAPSIBLE INTRAVAGINAL DEVICE FOR SELF-SAMPLING AND CONTAINMENT OF CERVICAL EPITHELIAL CELLS

(75) Inventor: Ramses Galaz Mendez, Hermosillo (MX)

(73) Assignee: ARX TECH LLC, Hermosillo, Sonora (MX)

(21) Appl. No.: 13/392,819

(22) PCT Filed: Aug. 20, 2010
(86) PCT No.: PCT/IB10/02057

§ 371 Date: Feb. 27, 2012

(30) Foreign Application Priority Data
Aug. 28, 2009 (MX) MX-A-2009-9553

(51) Int. Cl. A61B 10/02

(52) U.S. Cl. 600/562

(57) ABSTRACT

A device is described that allows women to collect and preserve cervical cell samples to be later tested in a clinical analysis laboratory. It includes a cylindrical body, housing a shaft that axially pushes and retracts a cell collector. The penetration necessary for the insertion device is self-induced by the woman, and therefore has morphological characteristics that are adaptable to the anatomical shape of the vagina. The shaft slides inside the device to position the cell collector on the surface of the cervix. Once the sample is taken, the shaft is retracted, and the cell collector collapses as a consequence of the sliding inside the device, and positioning itself inside the handle, which may contain a liquid medium for cellular containment.

### FIELD OF THE INVENTION

This invention concerns a device for sampling cervical epithelial cells, which complies with practical functions of devices for analogous purposes used currently in the medical industry. With this invention notable improvements have been obtained in relation to previously known devices of this type. The device is adaptable to the anatomical form of the neck of the uterus, is easy to handle, and achieves a comfortable and safe collection and containment of said cervical epithelial cells within the same device.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Well-known studies have been carried out by sampling cervical cells to detect pre-tumorous lesions in the neck of the uterus. These pre-tumorous lesions are associated with cervical cancer, an illness that is the primary cause of death in women after the age of twenty-five. These studies should be carried out periodically; according to international health standards, they should be repeated every year. By using this exam periodically in the target population, it is believed that 90% of deaths associated with this illness may be prevented worldwide.

Unfortunately, there are economic, cultural, social and other factors that make it unlikely that this testing will take place in the majority of the worldwide population; especially in developing countries, (i) the financial cost and investment of time involved in going to an appointment with a gynecologist or health center, (ii) the low or non-existent confidence that patients have in medical or technical personnel and their facilities, (iii) the lack of information about the importance of testing, (iv) the embarrassment or shame in being so examined, and (v) the inconvenience involved in submitting oneself to this type of procedure as many conventional instruments used for this type of sampling are capable of inflicting pain, irritation, discomfort, and possible damage to the tissues of the cervix. Other factors contributing to the notable aversion that the female population has for these procedures concerns the possible risk that is inherent with sampling instruments poorly used by unskilled personnel and the record of lesions to the uterine neck in patients as a consequence of poorly executed procedures.

Some sampling devices have been developed to address at least some of the inconveniences mentioned above, and that allow a woman to take cervical cell samples in the comfort of her home with privacy. Such is the case in Mexican Patent Number MX 207449, that describes the insertion within the vagina of a device having a barrel with a sponge and a brush with a plunger. On its tip is a circular sponge positioned for collection of cervical tissue and mucous, placed in a retracted position within the barrel, and that upon exiting the sponge expands to collect the sample. After having collected the sample, the sponge and brush are removed from the shaft of the plunger, and within a few seconds afterwards the sample should be deposited in a container and then sealed and sent to a laboratory for analysis.

The aforementioned device serves as a base to highlight certain of the innovative characteristics of the present invention, among which are that one part of the vaginal insert is included that comprises a diameter that minimizes the need for vaginal canal expansion, which represents an aspect of convenience for the patient. Another advantage is an anatomical form that simplifies self-positioning of the cell collector to maximize the area of contact of the same with the transformation zone of the uterine neck, eliminating the propensity towards errors as a result of collection in areas that do not correspond to the cervix. The anatomical design and flexibility of the components of the device minimize pain and possible lesions to the cervical tissue, because they do not contain rigid and piercing or cutting parts that might penetrate, injure, or cause damage to the female anatomy. The cell collector is designed to have a layout of multiple protrusions making its surface rough to the touch. These protrusions are useful in maximizing the retention of the cervical mucus through viscous adhesion and surface tension of the fluid itself, which is beneficial, since the larger the amount of cells collected the better the analysis of the sample. The hydrophobic characteristics of the cell collector's material allow the cellular content to be easily released by diluting it in a liquid medium so that it may be later studied, unlike a hydrophilic or absorbent collector such as a sponge.

### SUMMARY OF THE INVENTION

In an embodiment, the invention includes a device for self-sampling and containment of cervical epithelial cells, comprising: a handle; an insertion segment affixed to the handle; a shaft slidably positioned through the handle and the insertion segment; and a cell collector mounted on the shaft. The device may include a flexible member affixed to the insertion segment configured to adapt to and accommodate the internal anatomy of the vaginal canal and cervix during use of the device. The device may include a knob positioned between the handle and the insertion segment. The knob may have a larger diameter than both the handle and the insertion segment. The device may include a vessel inside the handle, being configured to contain a volume of a liquid. The vessel may be bounded on its end furthest from the insertion segment by a cap, the cap being removably attached to the handle. The cap may further comprise a cap opening, in which the shaft is slidably positioned. The cap opening may be a valve configured to minimize or prevent the leakage of the liquid from the vessel and to close to a substantially liquid-tight state upon removal of the shaft therefrom. The cap opening may be configured to permit slidable passage of the shaft therethrough but not to permit passage of the cell collector therethrough. The vessel may be bounded on its end closest to the insertion segment by a membrane. The membrane may include a membrane opening, in which the shaft is slidably positioned. The membrane opening may be a valve configured to minimize or prevent the leakage of the liquid from the vessel and to close to a substantially liquid-tight state upon removal of the shaft therefrom. The membrane opening may be configured to permit slidable passage of both the shaft and the cell collector therethrough. The membrane opening may be configured to permit slidable passage of the cell collector therethrough, but only when traveling in a direction going from the insertion segment towards the handle. The cell collector may be removably mounted to the shaft. The cell collector may be configured to expand to a state in which it has a diameter in the range from 10 mm to 35 mm. The cell collector may be configured to expand to a state in which it has a diameter of about 25 mm. The cell collector may have a generally concave shape when deployed. The cell collector may include a flexible neck. The cell collector may include at least three flexible arms joined together by a flexible interdigital membrane. The cell collector may include six flexible arms joined together by a flexible interdigital membrane. The at least three flexible arms may each include a nub at their respective ends. The cell collector may include a center protrusion to at least partially enter the endocervical canal during use. The cell collector may include multiple bumps on its surface.

In another embodiment, the invention includes a method for sampling cervical cells, comprising: providing a device, comprising a handle, an insertion segment affixed to the handle, a shaft slidably positioned through the handle and the insertion segment, and a cell collector mounted on the shaft; inserting the insertion segment into the vaginal canal, whereby the insertion segment contacts the cervix; exposing the cell collector to the cervix, whereby cervical cells are removed from the cervix; and withdrawing the device from the vaginal canal. The method may be performed by a user to sample the user's own cervical cells. The device may include a flexible member affixed to the insertion segment, and wherein, upon insertion of the insertion segment into the vaginal canal, the flexible member may adapt to and accommodate the internal anatomy of the vaginal canal and cervix. The device may include a knob positioned between the handle and the insertion segment, wherein the knob has a larger diameter than both the handle and the insertion segment. The device may further include a vessel that contains a volume of a liquid, and the method may further comprise withdrawing the shaft and cell collector through the insertion segment and the handle, whereby the shaft is removed entirely from the device and the cell collector remains in the vessel.

### BRIEF DESCRIPTION OF THE DRAWINGS

For better understanding of that which is described herein, drawings are attached (which only serve as an example), showing a practical case of an embodiment of the anatomically designed and collapsible intravaginal device for self-sampling and containment of cervical epithelial cells. Like numerals represent like parts in the figures.

FIG. 1 is a front view of the anatomically designed and collapsible intravaginal device for self-sampling and containment of cervical epithelial cells, in accordance with an embodiment of the present invention.

FIG. 2 is an exploded isometric view of all the components of the anatomically designed and collapsible intravaginal device for self-sampling and containment of cervical epithelial cells, in accordance with an embodiment of the present invention.

FIG. 3 is a detailed view of the proximal end which that illustrates the anatomically designed and collapsible intravaginal device for self-sampling and containment of cervical epithelial cells in an expanded position, in accordance with an embodiment of the present invention.

FIG. 4 is a back view, that illustrates the anatomically designed and collapsible intravaginal device for self-sampling and containment of cervical epithelial cells in an expanded position, in accordance with an embodiment of the present invention.

FIG. 5 is a sagittal view which shows a cross-cut view of how the anatomically designed and collapsible intravaginal device for self-sampling and containment of cervical epithelial cells is introduced into the vagina, in accordance with an embodiment of the present invention.

FIG. 6 is a sagittal view which shows a cross-cut view of the deformability of the proximal part of insertion of the anatomically designed and collapsible intravaginal device for self-sampling and containment of cervical epithelial cells, to begin to adapt itself to the anatomical shape of the cervix, in accordance with an embodiment of the present invention.

FIG. 7 is a sagittal view which shows a cross-cut view of the procedure of positioning the cell collector on the anatomical surface of the cervix where the central; shaft may be seen pushing the cell collector towards the outside, causing it to expand to its normally relaxed position and it positions itself thusly, over the uterine neck, in accordance with an embodiment of the present invention.

FIG. 8 is a sagittal view that shows a cross-cut view of the procedure of retracting the central shaft that in turn shows the cell collector in a collapsed position within the anatomically designed and collapsible intravaginal device for self-sampling and containment of cervical epithelial cells, in accordance with an embodiment of the present invention.

FIGS. 9A, 9B and 9C are cross-sectional views of the device of FIG. 1, illustrating the sequence of events after the device is withdrawn from the vagina, and the cell collector is drawn through the device and deposited into the vessel within the handle, in accordance with an embodiment of the present invention.

FIG. 10 is an elevational view of the membrane and membrane opening, with a valve, in accordance with an embodiment of the present invention.

### DETAILED DESCRIPTION OF THE INVENTION

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Cancer” and “cancerous” refer to or describe the physiological condition in mammals that is typically characterized by unregulated cell growth. Embodiments of the present invention are useful in connection with a screening test for cervical cancer, a disease in which the cells of the cervix become abnormal and start to grow uncontrollably, forming tumors. The aforementioned screening test may be useful in human females of all ages.

The invention relates to a device and methods of use thereof for sampling cervical epithelial cells. The device provides practical and innovative methods to achieve streamlined and safe self-collection and immediate containment of endocervical cells in an environment that is comfortable for the test subject, eliminating the pain and risk that regularly inhibit the performance of these medical tests worldwide in the female population.

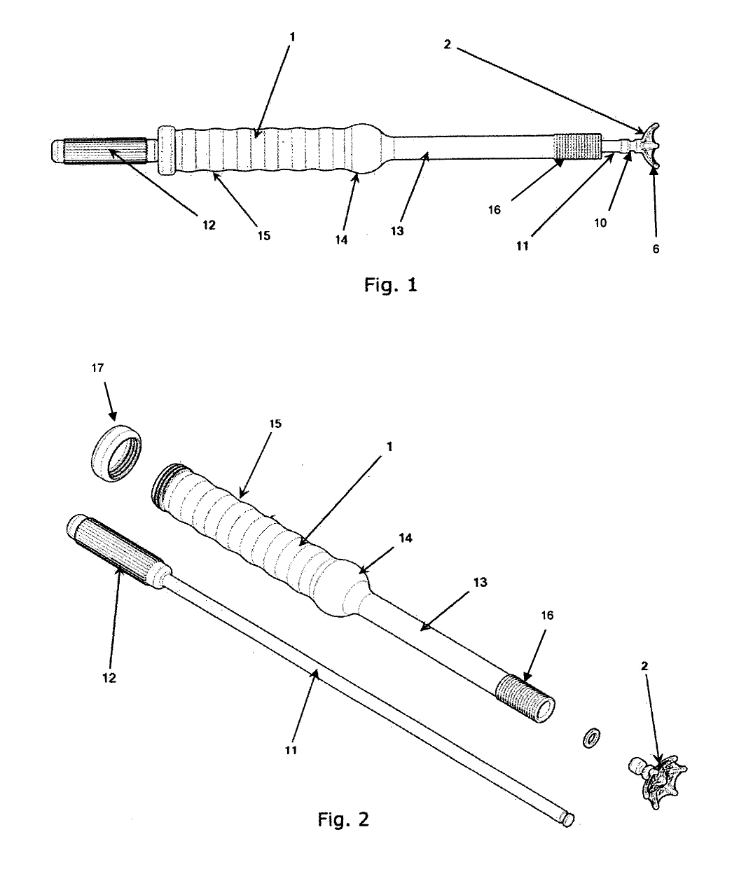

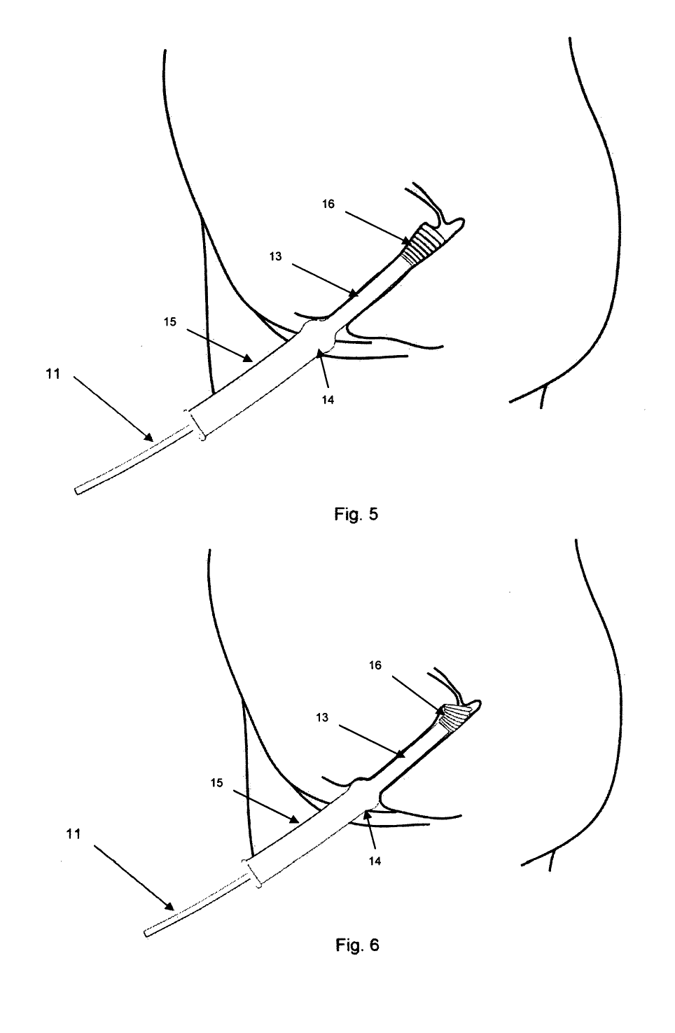

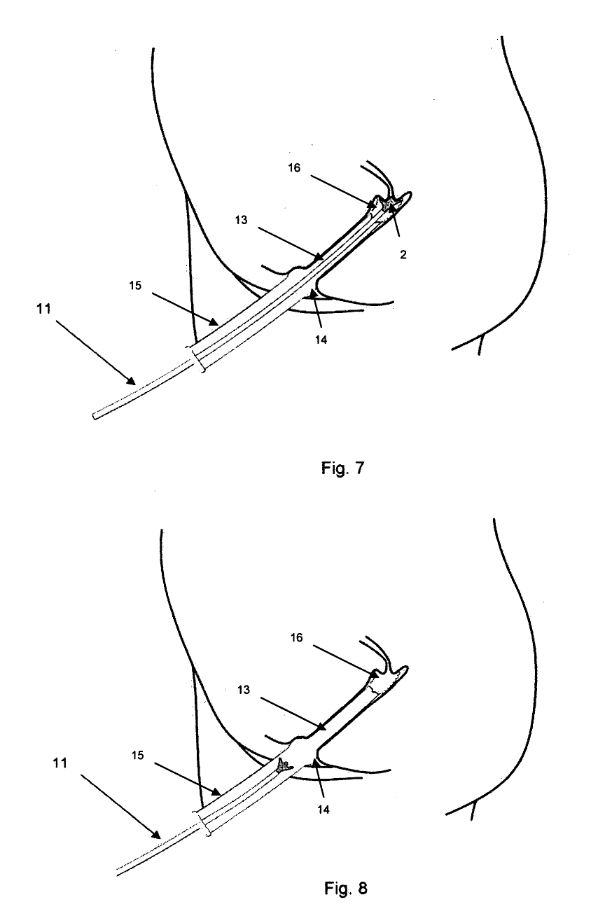

With reference to FIG. 1, in an embodiment the device 1 includes an insertion segment 13 and a handle 15. The insertion segment 13 may have a generally cylindrical configuration, although other configurations may be used in connection with alternate embodiments of the device. The insertion segment 13 may be substantially transparent, or in alternate embodiments of the invention, it may be opaque. The insertion segment 13 may be configured to be substantially linear along its longitudinal axis. At its distal end, the insertion segment 13 may include a flexible member 16 that is constructed of material and/or in a mechanical configuration that enables it to adapt to and accommodate the internal anatomy of the vaginal canal and cervix during use, as illustrated in FIGS. 5-8; for instance, it may be constructed of a generally flexible material that allows it to compress and/or bend and/or flex and/or widen, as may be desirable.

The device 1 may include a knob 14 configured between the insertion segment 13 and the handle 15. The knob 14 may have a diameter greater than the insertion segment 13 and/or greater than the handle 15. The knob 14 is configured to act as a stop to restrict axial movement of the hand of a user from the handle 15 towards the insertion segment 13. The knob 14 is also configured to physically limit the device from being introduced excessively into the vaginal canal; that is, the knob 14 may prevent the device from being inserted beyond the length of the insertion segment 13 (see, for example, FIGS. 6-8).

The handle 15 may be integrally molded with the device 1, and may be characterized by being the least flexible area of the device 1 and/or for having the greatest diameter (except, in some embodiments, for the diameter of the knob 14). The external surface of the handle 15 may be configured with a rough texture that allows greater holding power or an improved and/or comfortable grip for the user. Those of skill in the art will recognize a number of configurations for the external surface of the handle 15 that may be desirable to improve a user's ability to comfortably hold and operate the device 1; for instance, the handle may be contoured to accommodate the fingers of a user's hand.

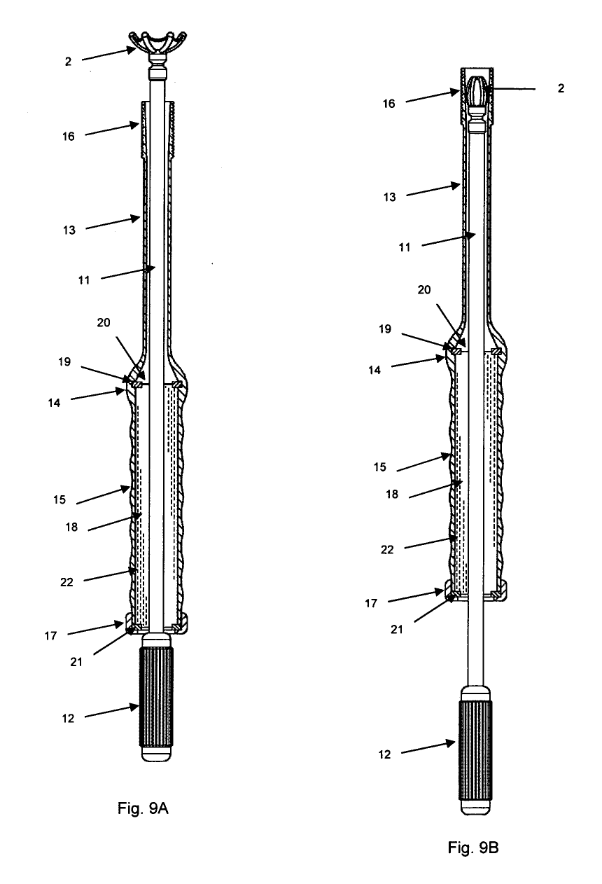

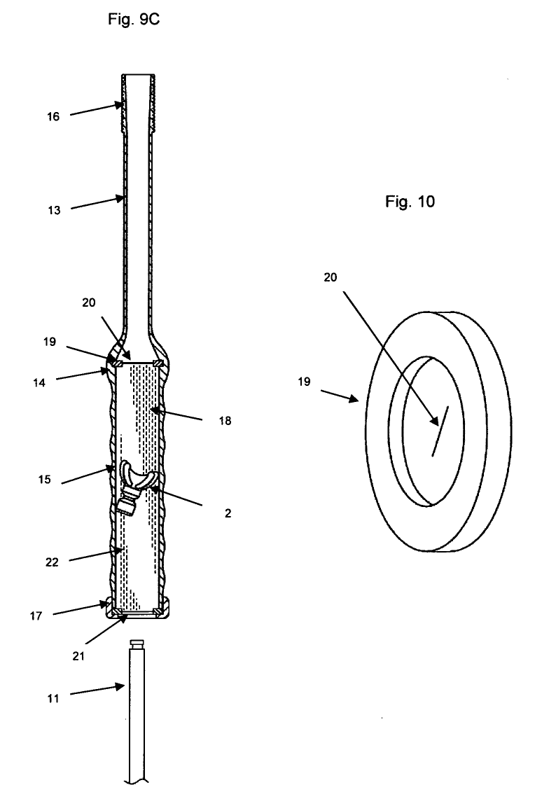

With reference to FIG. 9, the interior of the handle 15 may include a vessel 18 bounded by a cap 17 on one end and a membrane 19 on the other end. The vessel 18 is configured to contain a cellular suspension liquid suitable for liquid-based cytology and Hybrid-Capture studies. In other words, the vessel 18 may contain a liquid 22 suitable for storage of a specimen that is harvested from a female user until the specimen is later removed for analysis. The interior surface of the handle 15 may serve as the exterior surface of the vessel 18, although in alternate embodiments (not shown) a roughly cylindrical insert may be included within the handle to establish the cylindrical walls of the vessel 18.

The cap 17 includes a cap opening 21 through which the shaft 11 is able to slide. The cap opening 21 may be configured at or near the axial center of the cap 17. The cap opening 21 may be configured to enable passage of the shaft 11 therethrough, while minimizing or preventing the leakage of liquid 22 from the vessel 18. The cap 17 may be affixed to the handle 15 by any suitable mechanical arrangement; for instance, as illustrated in the drawings, with corresponding interior and exterior screw threading. Thus, after use of the device 1 to collect a sample of endocervical cells, the cap 17 may be removable by a technician in order to extract the sample.

Similar to the cap 17, the membrane 19 includes a membrane opening 20 through which the shaft 11 is able to slide. The membrane opening 20 may be configured at or near the axial center of the membrane 19. The membrane opening 20 may be configured to enable passage of the shaft 11 therethrough, while minimizing or preventing the leakage of liquid 22 from the vessel 18. The membrane 19 may be constructed of any suitable materials, and may be solid, rigid, semi-rigid, or flexible.

The membrane opening 20 and cap opening 21 may be configured as valves that enable the passage of mechanical elements therethrough, but that close in a liquid-tight state once such mechanical elements no longer reside within them. For example, the membrane opening 20 and cap opening 21 may allow for the shaft 11 to slide through them, but then close to a liquid-tight state once the shaft 11 slides completely through the membrane opening 20 and cap opening 21. Furthermore, the membrane opening 20 may also accommodate the passage of the cell collector 2 through it, while the cap opening 21 may not. Thus, in operation (as illustrated in FIGS. 9A-9C), when the shaft 11 coupled to cell collector 2 is slid through the device 1 (e.g., following collection of a specimen from a user), the shaft 11 and cell collector 2 may slide through the membrane opening 20, but then the shaft 11 may slide completely through the cap opening 21 while the cell collector 2 separates from the shaft 11, remaining within the vessel 18. In an embodiment, a membrane check valve is used for this purpose in the membrane opening 20 and/or in the cap opening 21.

The shaft 11 may be a solid, generally cylindrical (although other shapes may be suitable in alternate embodiments), flexible element. In an embodiment, it may be constructed of any number of materials, such as a high density polyethylene polymer or another polymer of a similar consistency. The shaft 11 may include a shaft handle 12, configured to facilitate a user pushing, retracting and/or rotating the shaft 11. In an embodiment, the shaft handle 12 includes lengthwise grooves to promote a better grip by a user, but, as with the handle 15, those of skill in the art will recognize a number of configurations for the shaft handle 12 that may be desirable to improve a user's ability to comfortably hold and operate it.

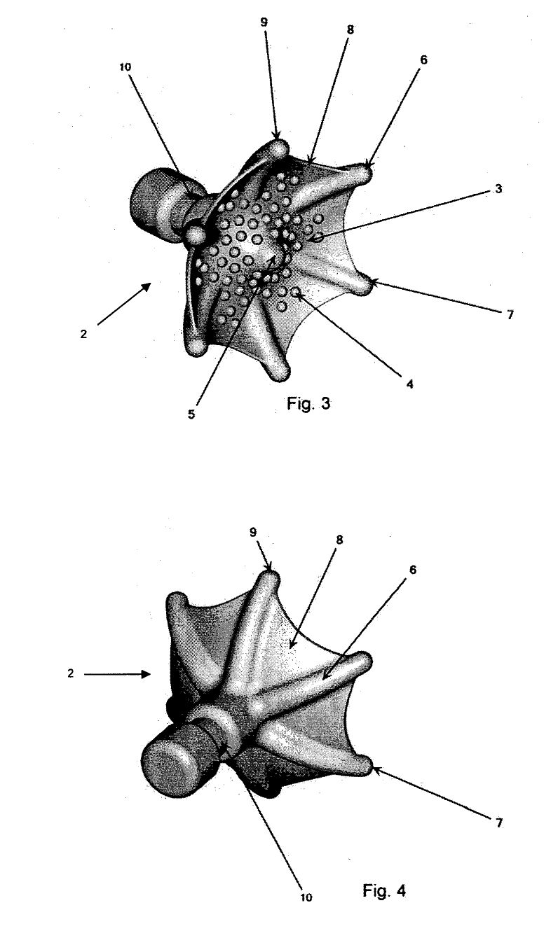

The shaft 11 may be integrally molded with, or, alternatively, removably coupled to the base of the cell collector 2. This may be accomplished by any number of mechanisms, such as an adhesive, a mechanical attachment, or any other method suitable to couple the two elements together. With reference to FIGS. 3 and 4, the cell collector 2 is configured geometrically with the ability to collapse and slide inside the insertion end 13 of the device 1. It is also configured to expand to an expanded state, in which the cell collector 2 may have a diameter of at least about 25 mm, and may thus generally cover the majority of the anatomical areas of the cervix when deployed. In alternate embodiments, the expansion size of the cell collector 2 may differ, and may instead be approximately 10 mm, 15 mm, 20 mm, 30 mm, 35 mm or any size therebetween. The cell collector 2 may include a generally concave form when deployed, that, owing to the flexibility of the cell collector 2, may mold itself to the various anatomical areas of the cervix.

The cell collector 2 may include a flexible neck 10, enabling additional flexibility when positioning in the area of the cervix. It may include at least three flexible arms 6 joined together by an interdigital membrane 8 that is also flexible. In alternate embodiments, the cell collector 2 may have 4, 5, 6, 7, 8 or more flexible arms 6. Thus, the cell collector 2 may expand to a relaxed position, permitting it to adapt to the anatomy of the cervix once it is outside of the insertion end 13 of the device 1. Each of the flexible arms 6 may include a rounded nub 7 at its tip. The nubs 7 (as well as nubs 6 and 9, for illustration purposes) may prevent or at least lessen the risk that the tissue of the uterine neck may suffer lesions or severe lesions during use of the device 1.

The collection surface 3 of the cell collector 2, which is in direct contact with organic tissue when in use, may have a center protrusion 5 in a generally conical or “bullet-shaped” form that lacks sharp forms on its tip and that is configured to at least partially enter the endocervical canal upon deployment. The collection surface 3 may further include multiple bumps 4, which in one embodiment are hemispheroidal, that are distributed about the center protrusion 5. The multiple bumps 4 may be positioned to scrape the area known to physicians as the “transformation zone of the cervix.” The multiple bumps 4 may be distributed randomly or in any advantageous pattern, as will be readily appreciated by those of skill in the art. The multiple bumps 4 may be capable of trapping a greater amount of fluid and/or cells due to viscous effects and surface tension of the fluid, than would a collection surface 3 that is smooth (although which is also within the scope of the present invention). In alternate embodiments of the invention, the collection surface 3 may be constructed of any number of different flexible materials, with or without multiple bumps 4 or analogous structures, but in each case configured to accomplish the task of enabling the collection of endocervical cells. The nubs 7 and multiple bumps 4 may be useful in maximizing the retention of the cervical mucus through viscous adhesion and surface tension of the fluid itself, which is beneficial, since the larger the amount of cells collected the better the ultimate analysis of the sample. The hydrophilic characteristics of the cell collector material may also allow the cellular content to be easily released by diluting it in a liquid medium for its testing afterwards, unlike a hydrophilic or absorbent collector such as a sponge.

In an embodiment, with reference to FIGS. 5-9, the device 1 may be operated as follows. The device 1 is provided, with the shaft 11 residing within it, and with the cell collector 2 collapsed within the insertion end 13. The insertion end 13 of the device 1 is introduced into the vagina. The insertion end 13 and the flexible member 16 at its tip mold and contour themselves to the anatomical form of the vaginal canal and of the cervix. For instance, the insertion end 13 and flexible member 16 may flex to accommodate a curvature of the vaginal canal and the anatomy at the junction between the vaginal canal and the cervix. Moreover, as illustrated in FIG. 6, the flexible member 16 may widen axially as it presses against the cervix.

Once the device 1 reaches the cervix and the flexible member 16 is configured against it, the user pushes and rotates the shaft handle 12 to expand the cell collector 2, thereby expanding it to a relaxed position on the uterine neck, where it molds itself anatomically thereto. The user may further rotate the shaft handle 12, causing the cell collector 2 to rotate upon the neck of the uterus. Multiple bumps 4 on the cell collector 2 facilitate exfoliation of cells in the transformation zone of the cervix and the retention of cervical fluids and cells. Once the sample is collected, the device 1 is removed from the vagina and the user retracts the shaft 11, which in turn withdraws the cell collector 2 and it collapses towards the inside of the insertion end 13 of the device 1.

The user further withdraws the shaft 11 from the device, such that the cell collector 2 retreats through the insertion end 13 and through the membrane opening 20 into the vessel 18. The user completely withdraws the shaft 11 from the device 1 through the cap opening 21, and in so doing, the shaft 11 separates from the cell collector 2, which remains within the vessel 18, suspended in the liquid 22. The aforementioned procedure allows for rapid immersion of the cell collector 2 into the liquid 22, minimizing exposure of the sampled cells and mucous to the air of the environment for long periods, which might damage the quality thereof due to the loss of moisture. The total enclosure of the cell collector 2 once the sample has been taken may also prevent the sample from being contaminated with fluids or cells from the vaginal wall and which would not be of any interest for the analysis.

The device 1 is then sent to a laboratory for analysis of the collected specimen. A laboratory technician removes the cap 17 from the handle 15, whereby the cell collector 2 and/or any cells or other sample materials collected and immersed in the liquid 22 can be accessed and analyzed.

The device 1 may be disposable in its entirety. This prevents the risk of containing infectious agents that may contaminate the patient, since each specimen will be used only one time and in one single event, after which it will be rendered useless and must be disposed of.

Various embodiments of the invention are described above. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the inventor at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the subject matter.

While particular embodiments of the present invention have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A device for self-sampling and containment of cervical epithelial cells, comprising:
a handle;
an insertion segment affixed to the handle;
a shaft slidably positioned through the handle and the insertion segment; and
a cell collector mounted on the shaft.

2. The device of claim 1, further comprising a flexible member affixed to the insertion segment, said flexible member being configured to adapt to and accommodate the internal anatomy of the vaginal canal and cervix during use of the device.

3. The device of claim 1, further comprising a knob positioned between the handle and the insertion segment.

4. The device of claim 3, wherein the knob has a larger diameter than both the handle and the insertion segment.

5. The device of claim 1, further comprising a vessel inside the handle, said vessel being configured to contain a volume of a liquid.

6. The device of claim 5, wherein the vessel is bounded on its end furthest from the insertion segment by a cap, said cap being removably attached to the handle.

7. The device of claim 6, wherein the cap further comprises a cap opening, in which the shaft is slidably positioned.

8. The device of claim 7, wherein the cap opening is a valve configured to minimize or prevent the leakage of the liquid from the vessel and to close to a substantially liquid-tight state upon removal of the shaft therefrom.

9. The device of claim 7, wherein the cap opening is configured to permit slidable passage of the shaft therethrough but not to permit passage of the cell collector therethrough.

10. The device of claim 5, wherein the vessel is bounded on its end closest to the insertion segment by a membrane.

11. The device of claim 10, wherein the membrane further comprises a membrane opening, in which the shaft is slidably positioned.

12. The device of claim 11, wherein the membrane opening is a valve configured to minimize or prevent the leakage of the liquid from the vessel and to close to a substantially liquid-tight state upon removal of the shaft therefrom.

13. The device of claim 11, wherein the membrane opening is configured to permit slidable passage of both the shaft and the cell collector therethrough.

14. The device of claim 13, wherein the membrane opening is configured to permit slidable passage of the cell collector therethrough, but only when traveling in a direction going from the insertion segment towards the handle.

15. The device of claim 1, wherein the cell collector is removably mounted to the shaft.

16. The device of claim 1, wherein the cell collector is configured to expand to an expanded state, in which the cell collector has a diameter in the range from 10 mm to 35 mm.

17. The device of claim 1, wherein the cell collector is configured to expand to an expanded state, in which the cell collector has a diameter of about 25 mm.

18. The device of claim 1, wherein the cell collector has a generally concave shape when deployed.

19. The device of claim 1, wherein the cell collector further comprises a flexible neck.

20. The device of claim 1, wherein the cell collector further comprises at least three flexible arms joined together by a flexible interdigital membrane.

21. The device of claim 1, wherein the cell collector comprises six flexible arms joined together by a flexible interdigital membrane.

22. The device of claim 20, wherein the at least three flexible arms each include a nub at their respective ends.

23. The device of claim 1, wherein the cell collector further comprises a center protrusion to at least partially enter the endocervical canal during use.

24. The device of claim 1, wherein the cell collector further comprises multiple bumps on its surface.

25. A method for sampling cervical cells, comprising:
providing a device, comprising
a handle,
an insertion segment affixed to the handle,
a shaft slidably positioned through the handle and the insertion segment, and
a cell collector mounted on the shaft;
inserting the insertion segment into the vaginal canal, whereby the insertion segment contacts the cervix;
exposing the cell collector to the cervix, whereby cervical cells are removed from the cervix; and
withdrawing the device from the vaginal canal.

26. The method of claim 25, wherein the method is performed by a user to sample the user's cervical cells.

27. The method of claim 25, wherein the device further comprises a flexible member affixed to the insertion segment, and wherein, upon said insertion of said insertion segment into the vaginal canal, said flexible member adapts to and accommodates the internal anatomy of the vaginal canal and cervix.

28. The method of claim 25, wherein the device further comprises a knob positioned between the handle and the insertion segment, and wherein the knob has a larger diameter than both the handle and the insertion segment.

29. The method of claim 25, wherein the device further comprises a vessel that contains a volume of a liquid, and the method further comprises withdrawing the shaft and cell collector through the insertion segment and the handle, whereby the shaft is removed entirely from the device and the cell collector remains in the vessel.
